(19) United States
(12) Patent Application Publication
(10) Pub. No.: US 2013/0138221 A1
(43) Pub. Date: May 30, 2013

(54) METHODS AND COMPOSITIONS FOR IMPROVING IMPLANT OSSEOINTEGRATION

(71) Applicants: Uwe Junker, Basel (CH); Michaela Kneissel, Basel (CH); Ina Kramer, Basel (CH); Falko Schlottig, Fullinsdorf (CH)

(72) Inventors: Uwe Junker, Basel (CH); Michaela Kneissel, Basel (CH); Ina Kramer, Basel (CH); Falko Schlottig, Fullinsdorf (CH)

(73) Assignee: Novartis AG, Basel (CH)

(21) Appl. No.: 13/641,506

(22) PCT Filed: Apr. 14, 2011
(86) PCT No.: PCT/EP11/55970

§ 371 Date: Oct. 16, 2012

Related U.S. Application Data
(60) Provisional application No. 61/324,901, filed on Apr. 16, 2010.

(51) Int. Cl. A61L 27/34

(52) U.S. Cl. CPC A61L 27/34
USPC 623/23.57

(57) ABSTRACT

The disclosed methods, uses and articles are in the field of orthopedic and dental implants. In particular, the disclosure relates to compositions and methods for improving the osseointegration of such implants.

### TECHNICAL FIELD

The disclosed methods, uses and articles are in the field of orthopedic and dental implants. In particular, the disclosure relates to compositions and methods for improving the osseointegration of such implants.

### BACKGROUND OF THE DISCLOSURE

Injured or damaged parts of the hard and/or soft tissue of the human body are best restored or mechanically reinforced using autologous hard and/or soft tissue. However, this is not always possible, which is why synthetic material may be used as a temporary (biodegradeable or post-operatively removeable) or permanent replacement material.

Such implants may be used to repair hard and/or soft tissue which has been damaged by accident, abrasion, genetic deficiency or sickness. The implant may support or take over the role of the natural tissue. For example, hip and knee joint prostheses and spinal implants have been used for many years [1, 2]. However, the anchoring of the implant and implant tolerance at the interface between the implant surface and the neighbouring tissue is of critical importance.

The loosening of implants from bone tissues has been a cause of problems in reconstructive surgery and joint replacement. Osseointegration of orthopaedic and dental implants is the key factor used to determine success of implantation [3]. Not only does failure to osseointegrate cause cost implications due to the need to repeat procedures, but such failure also causes pain and suffering to the patients. For example, about 8% of maxillar and 5% of mandibular implants fail in the normal population. Screw loosening in long bones is reported to be in the range of 3-6.5%. If such screw loosening occurs in the hip of an elderly patient, such an event may lead to death due to complications of a second surgery to remedy the problem [4].

Various methods have been attempted to improve osseointegration of implants such as using different materials (e.g., titanium and its alloys), roughening the surface of the implant (e.g. by sand blasting or acid-etching) or by the addition of bioactive coatings to the implant (e.g. calcium phosphate, bisphosphonate or collagen). However, despite these various modifications, which all apparently improve osseointegration compared to an untreated titanium implant, there is no single outstanding method, with simple roughening providing a similar improvement to bioactive coatings [5].

There is therefore a need for further methods for improving the osseointegration of implants.

### SUMMARY OF THE DISCLOSURE

It has been discovered that osseointegration of a bone implant can be improved by using a combination of a bone resorption inhibitor (e.g., a bisphosphonate, such as zoledronic acid) and a bone anabolic agent (e.g., an anti-sclerostin antibody, such as Antibody 1, or PTH). While a bone resorption inhibitor (e.g., a bisphosphonate, such as zoledronic acid) alone may prevent further bone loss, it will not actively encourage bone growth. While a bone anabolic agent causes new bone growth, the effect may quickly diminish. However, the effect of a bone anabolic agent is enhanced and extended by the presence of the bone resorption inhibitor (e.g., a bisphosphonate, such as zoledronic acid). The methods and compositions of the invention may also be used to facilitate implantation and/or reduce the time required for osseointegration of a bone implant (i.e., to reduce the recovery time following a surgical procedure/placement of an implant), enhance osseointegration, prevent implant rejection and/or failure, and promote bone growth and development.

Thus, the disclosure provides, inter alia, a method for improving the osseointegration of a bone implant comprising administering at least one bone anabolic agent (e.g., at least one anti-sclerostin anbody, e.g., Antibody 1, or PTH) and at least one bone resorption inhibitor (e.g., at least one bisphosphonate, such as zoledronic acid) to the patient in receipt of said implant.

In another embodiment, the disclosure provides a combination of at least one bone anabolic agent (e.g., at least one anti-sclerostin anbody, e.g., Antibody 1, or PTH) and at least one bone resorption inhibitor (e.g., at least one bisphosphonate, such as zoledronic acid) for improving the osseointegration of a bone implant.

In one embodiment the bone anabolic agent (e.g., at least one anti-sclerostin anbody, e.g., Antibody 1, or PTH) is administered systemically and the bone resorption inhibitor (e.g., at least one bisphosphonate, such as zoledronic acid) is administered systemically. In one embodiment the bone anabolic agent (e.g., at least one anti-sclerostin anbody, e.g., Antibody 1, or PTH) is administered systemically and the bone resorption inhibitor (e.g., at least one bisphosphonate, such as zoledronic acid) is administered locally. In one embodiment the bone anabolic agent (e.g., at least one anti-sclerostin anbody, e.g., Antibody 1, or PTH) is administered locally and the bone resorption inhibitor (e.g., at least one bisphosphonate) is administered systemically. In one embodiment the bone anabolic agent (e.g., at least one anti-sclerostin anbody, e.g., Antibody 1, or PTH) is administered locally and the bone resorption inhibitor (e.g., at least one bisphosphonate, such as zoledronic acid) is administered locally.

In one embodiment the bone anabolic agent (e.g., at least one anti-sclerostin anbody, e.g., Antibody 1, or PTH) is coated onto the implant. In one embodiment the bone resorption inhibitor (e.g., at least one bisphosphonate, such as zoledronic acid) is coated onto the implant. In one embodiment, both the bone anabolic agent (e.g., at least one anti-sclerostin anbody, e.g., Antibody 1, or PTH) and the bone resorption inhibitor (e.g., at least one bisphosphonate, such as zoledronic acid) are coated onto the implant.

If they are both administered systemically, the bone anabolic agent (e.g., at least one anti-sclerostin anbody, e.g., Antibody 1, or PTH) and bone resorption inhibitor (e.g., at least one bisphosphonate, such as zoledronic acid) may be administered in either order.

If the bone anabolic agent (e.g., at least one anti-sclerostin anbody, e.g., Antibody 1, or PTH) is administered locally, the bone resorption inhibitor (e.g., at least one bisphosphonate, such as zoledronic acid) may be administered before or after the implant is fixed in place. Likewise, if the bone resorption inhibitor (e.g., at least one bisphosphonate, such as zoledronic acid) is administered locally, the bone anabolic agent may be administered before or after the implant is fixed in place.

Local administration may be achieved by a local injection, coating of the implant or by application of a local depot formulation. Thus, in one embodiment, the local administration may be applied directly into the bone marrow cavity of a bone (e.g. in the case of joint replacements), or as a filler around the implant once implanted.

In one embodiment, the disclosure provides a bone implant coated with a bone anabolic agent (e.g., at least one anti-sclerostin anbody, e.g., Antibody 1, or PTH) and/or a bone resorption inhibitor (e.g., at least one bisphosphonate, such as zoledronic acid). In one embodiment, the disclosure provides a bone implant coated with a bone anabolic agent (e.g., at least one anti-sclerostin anbody, e.g., Antibody 1, or PTH) and a bone resorption inhibitor (e.g., at least one bisphosphonate, such as zoledronic acid).

In one embodiment the bone resorption inhibitor is a bisphosphonate. In one embodiment the bone resorption inhibitor is a RANKL antibody (such as denosumab).

In one embodiment the bone anabolic agent is an anti-sclerostin antibody. In one embodiment, the anti-sclerostin antibody is Antibody 1, as disclosed in WO09047356, the contents of which are incorporated by reference herein in its entirety. In one embodiment the bone anabolic agent is parathyroid hormone (PTH), or a fragment of PTH.

In one embodiment, an anti-sclerostin antibody and a bisphosphonate are the sole active ingredients for use with the implant.

### DETAILED DESCRIPTION OF THE DISCLOSURE

### Bone Resorption Inhibitor

Bone resorption inhibitors suitable for use in the disclosed methods and implants include, but are not limited to, bisphosphonates (e.g., Fosamax™ (alendronate), Actonel™ (risedronate sodium), Boniva/Bonviva™ (ibandronic acid), Zometa™ (zoledronic acid), Aclasta™/Reclast™ (zoledronic acid), olpadronate, neridronate, etidronate, clodronate, skelid, bonefos), Selected Estrogen Receptor Modulators (SERMs, such as raloxifene, lasofoxifene, bazedoxifene, arzoxifene, FC1271, Tibolone (Livial®)), estrogen, strontium ranelate and calcitonin. In one embodiment, the bone resorption inhibitor is calcitonin (e.g., a salmon calcitonin (sCT), such as Miacalcin™). In yet a further embodiment, the sCT is administered orally in combination with a suitable oral carrier, such as those set forth in U.S. Pat. No. 5,773,647 (herein incorporated by reference in its entirety), e.g., 5-CNAC and pharmaceutically acceptable salts (e.g., the disodium salt of 5-CNAC) and esters thereof. In certain embodiments, sCT may be administered with PTH and the disodium salt of 5-CNAC. In one embodiment, the bone resorption inhibitor is a RANKL antibody. In one embodiment the RANKL antibody is denosumab.

### Bisphosphonate

The bisphosphonates used in the present methods and implants are those which inhibit bone resorption. Such compounds characteristically contain two phosphonate groups attached to a single carbon atom, forming a “P-C-P” structure, e.g. in a compound of formula I

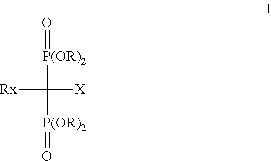

wherein
X is hydrogen, hydroxyl, amino, alkanoyl, or an amino group mono- or disubstituted by C₁-C₄ alkyl;
R is hydrogen or C₁-C₄ alkyl and
Rx is an optionally substituted hydrocarbyl group,
and pharmaceutically acceptable salts thereof or any hydrate thereof.

Thus, for example, suitable bisphosphonates for use in the disclosed methods and implants may include the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-ethidene-bisphosphonic acid, e.g. etidronate; 1-hydroxy-3-(methylpentylamino)-propylidene-bisphosphonic acid, (ibandronic acid), e.g. ibandronate; 6-amino-1-hydroxyhexane-1,1-diphos-phonic acid, e.g. amino-hexyl-BP; 3-(N-methyl-N-n-pentylamino)-1-hydroxypropane-1,1-di-phosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methylpyridinium salts thereof, for example N-methylpyridinium iodides such as NE-10244 or NE-10446; 1-(4-chlorophenylthio)methane-1,1-diphosphonic acid (tiludronic acid), e.g. tiludronate; 3-[N-(2-phenylthioethyl)-N-methyl-amino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); 1-hydroxy-2-(imidazo[1,2-a]pyridin-3-yl)ethane-1,1-diphosphonic acid, e.g. YM 529; and 1,1-dichloromethane-1,1-diphosphonic acid (clodronic acid), e.g. clodronate; YM175.

In one embodiment, bisphosphonates used in the present methods and implants are N-bisphosphonates, i.e. compounds which in addition to the characteristic geminal bisphosphonates moiety (e.g. “P-C-P”) comprise a nitrogen-containing side chain, e.g. a compound of formula I′

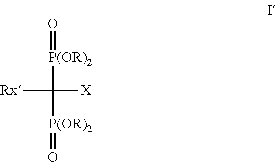

wherein
X is hydrogen, hydroxyl, amino, alkanoyl, or an amino group mono- or disubstituted by C₁-C₄ alkyl;
R is hydrogen or C₁-C₄ alkyl and
Rx′ is a side chain which contains an optionally substituted amino group, or a nitrogen containing heterocycle (including aromatic nitrogen-containing heterocycles),
and pharmaceutically acceptable salts thereof or any hydrate thereof.

Thus, for example, suitable N-bisphosphonates for use in the disclosed methods and implants may include the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methylpyridinium salts thereof, for example N-methylpyridinium iodides such as NE-10244 or NE-10446; 3-[N-(2-phenylthioethyl)-N-methylamino]-1-hydroxy-propane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); and 1-hydroxy-2-(imidazo[1,2-a]pyridin-3-yl)ethane-1,1-diphosphonic acid, e.g. YM 529.

In one embodiment an N-bisphosphonate for use in the disclosed methods and implants comprises a compound of Formula II

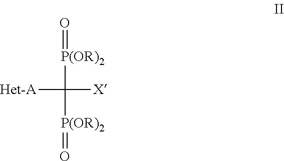

wherein
Het is an imidazole, oxazole, isoxazole, oxadiazole, thiazole, thiadiazole, pyridine, 1,2,3-triazole, 1,2,4-triazole or benzimidazole radical, which is optionally substituted by alkyl, alkoxy, halogen, hydroxyl, carboxyl, an amino group optionally substituted by alkyl or alkanoyl radicals or a benzyl radical optionally substituted by alkyl, nitro, amino or aminoalkyl;
A is a straight-chained or branched, saturated or unsaturated hydrocarbon moiety containing from 1 to 8 carbon atoms;
X′ is a hydrogen atom, optionally substituted by alkanoyl, or an amino group optionally substituted by alkyl or alkanoyl radicals, and
R is a hydrogen atom or an alkyl radical,
   and the pharmacologically acceptable salts thereof.

In a further embodiment a bisphosphonate for use in the disclosed methods and implants comprises a compound of Formula III

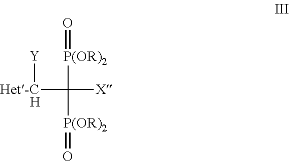

wherein
Het′ is a substituted or unsubstituted heteroaromatic five-membered ring selected from the group consisting of imidazolyl, imidazolinyl, isoxazolyl, oxazolyl, oxazolinyl, thiazolyl, thiazolinyl, triazolyl, oxadiazolyl and thiadiazolyl wherein said ring can be partly hydrogenated and wherein said substituents are selected from at least one of the group consisting of C₁-C₄ alkyl, C₁-C₄ alkoxy, phenyl, cyclohexyl, cyclohexylmethyl, halogen and amino and wherein two adjacent alkyl substituents of Het can together form a second ring;
Y is hydrogen or C₁-C₄ alkyl;
X″ is hydrogen, hydroxyl, amino, or an amino group substituted by C₁-C₄ alkyl, and
R is hydrogen or C₁-C₄ alkyl;
   as well as the pharmacologically acceptable salts and isomers thereof.

In a yet further embodiment a bisphosphonate for use in the disclosed methods and implants comprises a compound of Formula IV

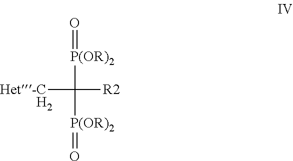

wherein
Het′″ is an imidazolyl, 2H-1,2,3-, 1H-1,2,4- or 4H-1,2,4-triazolyl, tetrazolyl, oxazolyl, isoxazolyl, oxadiazolyl, thiazolyl or thiadiazolyl radical which is unsubstituted or C-mono- or di-substituted by lower alkyl, by lower alkoxy, bx phenyl which may in turn be mono- or disubstituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen, and
R2 is hydrogen, hydroxy, amino, lower alkylthio or halogen,
lower radicals having up to and including 7 C-atoms,
   or a pharmacologically acceptable salt thereof.

Examples of N-bisphosphonates for use in the disclosed methods and implants are
2-(1-Methylimidazol-2-yl)-1-hydroxyethane-1,1-diphosphonic acid;
2-(1-Benzylimidazol-2-yl)-1-hydroxyethane-1,1-diphosphonic acid;
2-(1-Methylimidazol-4-yl)-1-hydroxyethane-1,1-diphosphonic acid;
1-Amino-2-(1-methylimidazol-4-yl)ethane-1,1-diphosphonic acid;
1-Amino-2-(1-benzylimidazol-4-yl)ethane-1,1-diphosphonic acid;
2-(1-Methylimidazol-2-yl)ethane-1,1-diphosphonic acid;
2-(1-Benzylimidazol-2-yl)ethane-1,1-diphosphonic acid;
2-(Imidazol-1-yl)-1-hydroxyethane-1,1-diphosphonic acid;
2-(Imidazol-1-yl)ethane-1,1-diphosphonic acid;
2-(4H-1,2,4-triazol-4-yl)-1-hydroxyethane-1,1-diphosphonic acid;
2-(Thiazol-2-yl)ethane-1,1-diphosphonic acid;
2-(Imidazol-2-yl)ethane-1,1-diphosphonic acid;
2-(2-Methylimidazol-4(5)-yl)ethane-1,1-diphosphonic acid;
2-(2-Phenylimidazol-4(5)-yl)ethane-1,1-diphosphonic acid;
2-(4,5-Dimethylimidazol-1-yl)-1-hydroxyethane-1,1-diphosphonic acid, and
2-(2-Methylimidazol-4(5)-yl)-1-hydroxyethane-1,1-diphosphonic acid,
   and pharmacologically acceptable salts thereof.

In one embodiment, the N-bisphosphonate for use in the disclosed methods and implants is 2-(imidazol-1yl)-1-hydroxyethane-1,1-diphosphonic acid (zoledronic acid) or a pharmacologically acceptable salt thereof.

Pharmacologically acceptable salts are preferably salts with bases, conveniently metal salts derived from groups Ia, Ib, IIa and IIb of the Periodic Table of the Elements, including alkali metal salts, e.g., potassium and especially sodium salts, or alkaline earth metal salts, preferably calcium or magnesium salts, and also ammonium salts with ammonia or organic amines.

Exemplary pharmaceutically acceptable salts are those where one, two, three or four, in particular one or two, of the acidic hydrogens of the bisphosphonic acid are replaced by a pharmaceutically acceptable cation, in particular sodium, potassium or ammonium, in first instance sodium.

Such an exemplary group of pharmaceutically acceptable salts is characterized by having one acidic hydrogen and one pharmaceutically acceptable cation, especially sodium, in each of the phosphonic acid groups.

The bisphosphonic acid derivatives mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hydroxy-2-(imidazol-1-yl)ethane-1,1-diphosphonic acid is prepared as described e.g. in U.S. Pat. No. 4,939,130.

As noted above, various bisphosphonates are known in the art and include, but are not limited to, Fosamax™ (alendronate), Actonel™ (risedronate sodium), Boniva/Bonviva™ (ibandronic acid), Zometa™ (zoledronic acid), Aclasta™/Reclast™ (zoledronic acid), olpadronate, neridronate, etidronate, clodronate, skelid, and bonefos.

In one embodiment, the bisphosphonate used in the disclosed methods and implants is a nitrogen-containing bisphosphonate. It is preferred that the bisphosphonate is zoledronic acid, such as Aclasta™/Reclast™.

Methods of dosing with bisphosphonates are disclosed in the art, such as in reference 18.

### Bone Anabolic Agents

Bone anabolic agents are agents that cause the active build up of new bone, rather than inhibiting the resorption of bone.

The bone anabolic agent may be an anti-sclerostin antibody (described in detail, below). Alternatively, the bone anabolic agent may be parathyroid hormone (PTH), a PTH fragment or a PTH derivative e.g. PTH (1-84) (such as Preos™), PTH (1-34) (such as Forteo™), PTH (1-36), PTH (1-38), PTH (1-31)NH2 or PTS 893. If PTH is administered as the bone anabolic agent, the systemic dosage will typically be about 20 μg or about 40 μg daily. In one embodiment the PTH is administered in a single daily dose. In a further embodiment, the PTH is administered in a twice daily dose. In certain embodiments, the PTH (e.g., PTH (1-36), PTH (1-38)) is administered orally in combination with a suitable oral carrier, such as those set forth in U.S. Pat. No. 5,773,647 (herein incorporated by reference in its entirety), e.g., N-(5-chlorosalicyloyl)-8-aminocaprylic acid (5-CNAC) and pharmaceutically acceptable salts (e.g., the disodium salt of 5-CNAC) and esters thereof.

### Anti-Sclerostin Antibody

Various anti-sclerostin antibodies have been disclosed in references 613, the contents of which are incorporated by reference herein in their entirety. Any of the antibodies disclosed in these references may be used in the disclosed methods and implants. In particular, an antibody comprising a heavy chain comprising SEQ ID NOs:245, 246 and 247 and a light chain comprising SEQ ID NOs:78, 79 and 80 of reference 13 may be used in the disclosed methods and implants. Other anti-sclerostin antibodies that may be used in the disclosed methods and implants include those known as AMG167 (www.clinicaltrials.gov/ct2/show/NCT00902356?term=AMG167&rank=1) and AMG785 (www.clinicaltrials.gov/ct2/results?term=AMG785).

A preferred antibody for use with the disclosed methods and implants is an anti-sclerostin antibody such as those disclosed in reference 14 (the complete contents of which are incorporated herein by reference). Particularly preferred is the antibody Antibody 1. Antibody 1 has a V_{H} domain with amino acid SEQ ID NO: 1 and a V_{L} domain with amino acid SEQ ID NO: 2. Other anti-sclerostin antibodies useful with the present disclosed methods and implants may include one or more (1, 2, 3, 4, 5 or 6) CDRs from Antibody 1. The CDRs in the heavy chain are SEQ ID NOs: 3, 4 & 5. The CDRs in the light chain are SEQ ID NOs: 6, 7 & 8. The Antibody 1 variable domains may be expressed as SEQ ID NOs: 9 and 10 to give a functional antibody, the Antibody 1 V_{H} CDRs may be expressed along with V_{H} framework regions (e.g., V_{H} human framework regions) to give a functional antibody, the Antibody 1 V_{L} CDRs may be expressed along with V_{L} framework regions (e.g., V_{L} human framework regions) to give a functional antibody, and Antibody 1 V_{H} and V_{L} CDRs may be expressed along with V_{H} and V_{L} framework regions (e.g., V_{H} and V_{L} human framework regions) to give a functional antibody (e.g., human or humanized).

As used herein, the term “antibody” means a polypeptide comprising a framework region from an immunoglobulin gene or fragments thereof that specifically binds and recognizes an epitope, e.g. an epitope found on sclerostin, as described above. Thus, the term antibody includes whole antibodies (such as monoclonal, chimeric, humanised and human antibodies), including single-chain whole antibodies, and antigen-binding fragments thereof. The term “antibody” includes antigen-binding antibody fragments, including single-chain antibodies, which can comprise the variable regions alone, or in combination, with all or part of the following polypeptide elements: hinge region, CH₁, CH₂, and CH₃ domains of an antibody molecule. Also included within the definition are any combinations of variable regions and hinge region, CH₁, CH₂, and CH₃ domains. Antibody fragments include, e.g., but are not limited to, Fab, Fab′ and F(ab′)₂, Fd, single-chain Fvs (scFv), single-chain antibodies, disulphide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulphide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989; Muyldermans et al., TIBS 24: 230-235, 2001), which consists of a V_{H} domain; and (vi) an isolated complementarity determining region (CDR). The term “antibody” includes single domain antibodies, maxibodies, minibodies, intrabodies, diabodies, triabodies, tetrabodies, v-NAR and bis-scFv (see, e.g., Hollinger & Hudson, Nature Biotechnology, 23, 9, 1126-1136 (2005)). Antigen binding portions of antibodies can be grafted into scaffolds based on polypeptides such as Fibronectin type III (Fn3) (see U.S. Pat. No. 6,703,199, which describes fibronectin polypeptide monobodies). Antigen binding portions can be incorporated into single chain molecules comprising a pair of tandem Fv segments (VH-CH1-VH-CH1) which, together with complementary light chain polypeptides, form a pair of antigen binding regions (Zapata et al., Protein Eng. 8(10):1057-1062 (1995); and U.S. Pat. No. 5,641,870).

Given that the antibodies used in the disclosed methods and implants can bind to sclerostin and that antigen-binding specificity is provided primarily by the CDR1, 2 and 3 regions, the VH CDR1, 2 and 3 sequences and VL CDR1, 2 and 3 sequences can be “mixed and matched” (i.e., CDRs from different antibodies can be mixed and matched), although each antibody must contain a VII CDR1, 2 and 3 and a VL CDR1, 2 and 3 to create other anti-sclerostin antibodies. Sclerostin binding of such “mixed and matched” antibodies can be tested using the binding assays described in WO2009/047356. When VH CDR sequences are mixed and matched, the CDR1, CDR2 and/or CDR3 sequence from a particular VII sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for monoclonal antibodies of the present disclosed methods and implants.

### Osseointegration

The terms osseointegration is used in this application to refer to both osseointegration and osteointegration. Typically the term “osseointegration” is used when used in the dental field and “osteointegration” is used when used in the spinal/long bone field as well as when referring to integration of replacement joints (such as, for example, hip, knee, shoulder, spine). However, both terms refer to the integration of the implant into the surrounding bone tissue.

The level of osseointegration of an implant can be determined by one of several methods. For example, the bone mineral density around an implant site, the area of bone/implant contact, bone volume, the force required to remove an implant, resonant frequency analysis and the torque required to remove the implant are all indicators of the level of osseointegration.

### Bone Mineral Density

Various methods for measuring bone mineral density are known in the art and include X-ray radiographs, Dual energy X-ray absorptiometry (DEXA), peripheral Dual energy X-ray absorptiometry (P-DEXA), dual photon absorptiometry (DPA), ultrasound, quantitative computed tomography (QCT), and Roentgen Stereophotogrammetry Analysis (RSA),” which can be used to study implant micromotion using implants with tantalum beads as “landmarks”. Improved osseointegration is said to be seen when the bone mineral density around the implant site is increased compared to a control implant where no bone anabolic agent or bone resorption inhibitor is present.

### Bone/Implant Contact Area

The area of an implant that is in contact with bone (bone/implant contact area) may be calculated using, for example, μCT (micro-computer tomography) or histomorphometry. Improved osseointegration is said to be seen when the area of implant in contact with bone is increased compared to a control implant where no bone anabolic agent or bone resorption inhibitor is present.

### Bone Volume

The volume of bone that grows such that it interleaves with the thread of a screw (i.e. between the screw pitch) or ribs on an implant may be measured. The greater the bone volume that interleaves with such a thread or ribs, the greater the stabilisation of the implant. Such bone volume may be calculated using, for example, μCT. Improved osseointegration is said to be seen when the volume of bone that interleaves with such a thread or ribs on an implant is increased compared to a control implant where no bone anabolic agent or bone resorption inhibitor is present.

As an alternative, bone volume within a certain radius of the implant can be measured.

### Torque Required to Remove Implant

Although only feasible in the experimental setting, the torque required to remove an implant can be measured by removing the implant with a torque spanner. Such a method is particularly used for screws or bolts. Improved osseointegration is said to be seen when the torque required to remove the implant is increased compared to a control implant where no bone anabolic agent or bone resorption inhibitor is present.

### Force Required to Remove Implant

Again, while only feasible in the experimental setting, the force required to pull or push an implant from a bone may be measured. Improved osseointegration is said to be seen when the force required to remove the implant is increased compared to a control implant where no bone anabolic agent or bone resorption inhibitor is present.

### Resonant Frequency Analysis

The resonant frequency of an implant can be measured to provide a relative readout of the stability of the implant. Once implanted, the implant may be excited by sonic or magnetic impulses. The resonant frequency of the implant may then be measured. A higher resonant frequency indicates a more stable implant. An example of such a measurement device is the Osstell ISQ™. Improved osseointegration is said to be seen when the resonant frequency of the implant is increased compared to a control implant where no bone anabolic agent or bone resorption inhibitor is present.

### Bone Implants

For the purposes of this disclosure, the term “bone implant” is considered to refer to both those implants that penetrate into the bone (e.g. bone screws), those that may only be found on the surface of the bone (e.g. bone plates, such as those used in assisting fracture healing) as well as those that bone grows into and replaces over time (such as collagen based implants—e.g., the Infuse® Bone Graft, which is a spinal implant combined with BMP2).

Various types of bone implants are known in the art and include bone plates, bone screws, dental implants, spinal implants and replacement joints, including, but not limited to knee, hip, ankle, shoulder, elbow, wrist and knuckle joints.

Various types of plates, pins and screws used with bone and fracture healing are known in the art, and various types are summarised in reference 15.

Included within the scope of the disclosed methods and implants are also those implants that allow prostheses (such as prosthetic noses, ears, legs, arms, fingers and thumbs) to be attached to the human body. Such implants have one end anchored in the bone, with the other end protruding through the skin.

Examples of such implants include the AEGIS™ Anterior Lumbar Plate System, the BENGAL™ Stackable Cage System, the CHARITÉ® Artificial Disc, the CONCORDE™ Bullet System, the DISCOVERY® Screw System, the EAGLE™ Plus Anterior Cervical Plate System, the EXPEDIUM® 4.5 Spine System, the EXPEDIUM® 6.35 Spine System, the EXPEDIUM® PEEK Rod System, the EXPEDIUM® SFX™ Cross Connector System, the MONARCH® 5.50 Ti Spine System, the MOSS® MIAMI SI Spine System, the MOUNTAINEER™ OCT Spinal System, the SKYLINE™ Anterior Cervical Plate System, the SUMMIT™ SI OCT System, the UNIPLATE™ Anterior Cervical Plate System, the VIPER™ System, the VIPER™2 Minimally Invasive Pedicle Screw System and the X-MESH™ Expandable Cage System by DePuy Spine; the PINNACLE® Hip Solutions with TRUEGLIDE™ technology, the SIGMA®Knee products, the GLOBAL® Shoulder products, and the ANATOMIC LOCKED PLATING SYSTEMS (A.L.P.S.) by DePuy Orthopaedics; the replacement hip, knee, elbow, shoulder products as well as the spinal and trauma products by Zimmer; the replacement hip and knee products as well as the hand, spinal and trauma products by Stryker; the trauma products, intervertebral disks and fixation systems by Synthes; and the hip, knee, shoulder and finger prostheses by Mathys.

Dental implants are introduced into the jaw in order to mount or fasten artificial teeth or prostheses.

Examples of such implants include the SPI® products from Thommen Medical; the various implants including the NobelActive™ and NobelReplace™ implants from Nobel Biocare; and the Straumann® Bone Level Implants from Straumann.

Such implants may be made out of a variety of materials or combinations of materials. For example, implants may be made from calcium-phosphate-ceramics, bioglass, glass-ceramics, calcium-carbonate, calcium-sulfate, organic polymers, pure titanium, titanium alloys, cobalt-chromium-alloys, stainless steel, collagen, gelatine, aluminium oxide (AlO₃), zirconium dioxide (ZrO₂), polyether-etherketone (PEEK), ultra high molecular weight polyethylene (UHMWPE or sometimes shortened to UHMW), materials of allogenic origin, materials of xenogenic origin or composites or mixtures of said materials.

The implant may have a treated or roughened surface in order to improve the integration with the neighbouring tissue (e.g. bone) and/or to speed up the healing process. Various methods for producing such surfaces are disclosed in e.g., reference 16. Other methods of chemically modifying the implant surface in order to improve osseointegration are known and are disclosed in, e.g., reference 17.

The implant surface may be porous or non-porous.

### Administration

### Systemic Administration

Systemic administration of the bone anabolic and/or bone resorption inhibitor may be achieved intravenously, intramuscularly, or subcutaneously. The bone anabolic and/or bone resorption inhibitor may be administered by injection or by infusion. If administered by infusion, the infusion may be administered over a period of 15 minutes or more. In some embodiments, the bone anabolic and/or bone resorption inhibitor may be delivered orally.

The bone anabolic agent and bone resorption inhibitor may be provided in separate containers and administered separately (but still simultaneously or sequentially). Alternatively, the bone anabolic agent and bone resorption inhibitor may be provided in the same container. For example, the bone anabolic agent and bone resorption inhibitor may be provided in a two- or three-compartment infusion set (bag) such as described in references 18, . . . 21.

The bone anabolic agent and bone resorption inhibitor may independently be provided as pre-concentrates to be diluted prior to administration, or as ready-to-use solutions. Alternatively, the bone anabolic agent and bone resorption inhibitor may be provided as lyophilisates. Furthermore, if the bone resorption inhibitor is a bisphosphonate, it may be provided as a fat emulsion or a dispersion. If dilution is required, then this should be done with a pharmaceutically acceptable diluent.

The bone anabolic agent and bone resorption inhibitor are preferably provided in one or more heat-sterilisable plastics containers.

The particular mode of administration and the dosage may be adjusted by the attending physician taking into account the particulars of the patient, especially age, weight, lifestyle, activity level, hormonal status (e.g. post-menopausal) and bone mineral density as appropriate.

If a bone resorption inhibitor, such as a bisphosphonate, is administered systemically, the dose may be from about 1 mg/yr to about 10 mg/yr, or about 2 mg/yr to about 8 mg/yr, or about 4 mg/yr to about 6 mg/yr. Such dosages particularly apply to more potent bisphosphonates, such as zoledronic acid when administered intravenously.

Other bone resorption inhibitors, such as bisphosphonates other than zoledronic acid are less potent (see table 1 of reference 22), but may be used in the co-treatment of the disclosed methods, albeit at higher doses (for example, zoledronic acid is 10,000 times more potent than etidronate). In such cases the dose may be about 1 mg/yr to about 50,000 mg/yr, or about 10 mg/yr to about 10000 mg/yr, or about 100 mg/yr to about 1000 mg/yr.

If an anti-sclerostin antibody (e.g., Antibody 1) is administered, the dose may be from about 1 mg/kg to about 500 mg/kg, or about 10 mg/kg to about 400 mg/kg, or about 100 mg/kg to about 350 mg/kg, or about 200 mg/kg to about 300 mg/kg.

For Antibody 1, the dose may be about 5 mg/kg to about 300 mg/kg, or about 10 mg/kg to about 200 mg/kg, or about 20 mg/kg to about 100 mg/kg, or about 30 mg/kg to about 50 mg/kg. In preferred embodiments, the antisclerostin antibody, e.g., Antibody 1, may be administered as about 20 mg/kg. In some embodiments, the antisclerostin antibody, e.g., Antibody 1, is administered daily, twice in a week, weekly, every other week, monthly, every other month, quarterly, every six months, or yearly. In some embodiments, the antisclerostin antibody, e.g., Antibody 1, is administered singly (i.e., only once) or multiply.

“mg/kg” means mg drug per kg body weight of the patient to be treated.

In one embodiment, the total dose of anti-sclerostin antibody given to a patient over the course of a year may be about 500 mg to about 50,000 mg, or about 1000 mg to about 10,000 mg.

If PTH is administered systemically as the bone anabolic agent, the dosage will typically be about 20 μg to about 40 μg daily, e.g., about 20 μg or about 40 μg daily.

### Local Administration

In one embodiment, the bone anabolic agent and/or bone resorption inhibitor may be administered by a local injection.

In one embodiment, the implant is coated with a bone anabolic agent and/or a bone resorption inhibitor. In one embodiment, the coating is a dry coating.

In a further embodiment, the bone anabolic agent may be administered by a local depot system. For example, the bone anabolic may be formulated and administered as a gel or jelly or other form of slow release depot system. Such a gel or jelly may be coated onto the implant prior to fixation of the implant. Alternatively, the gel or jelly may be administered to the cavity into which the implant will be fixed (e.g., a dental cavity in the jaw, femur prosthesis implantation site). Examples of such gels are found in reference 23 and U.S. Provisional Patent Application No. 61/379,522 (the contents of which are hereby incorporated by reference). In a further embodiment, the bone anabolic agent may be provided as a lyophilisate. In one embodiment, the bone anabolic agent is an anti-sclerostin antibody formulated as a gel as disclosed in reference 23 and U.S. Provisional Patent Application No. 61/379,522.

### Implant Coating

As disclosed above, in one embodiment, the implant may be coated with the bone anabolic (such as an anti-sclerostin antibody) and/or a bone resorption inhibitor (such as a bisphosphonate). The amount of bone anabolic/bone resorption inhibitor may vary depending on one or more of a number of factors including: (i) the size of the implant, (ii) the surface area of the implant, (iii) the location where the implant is to be implanted, (iv) any further complicating factors suffered by the patient (e.g. the patient may suffer from osteoporosis).

The coating may release the active agents (the bone resorption inhibitor and/or the bone anabolic agent) over a long or short period. Thus, the coating may release the active agents for about 6 months or less, about 3 months or less, about 1 month or less, about 2 weeks or less, about 1 week or less, about 3 days or less, or about 24 hours or less.

Of course, the implant could be prepared such that the bone anabolic agent and bone resorption inhibitor are released at different rates or for different periods of time. For example, the bone anabolic agent may be released over a longer period than the bone resorption inhibitor.

### Bisphosphonate Coating

Methods of coating bisphosphonates, such as zoledronic acid, onto implants has been previously described such as in references 24 and 25.

In one embodiment, salts of amino-bisphosphonates and long-chain carboxylic acids or long-chain alkane-sulfates, as well as said bisphosphonate-polymer salts can be applied to an implant as finely distributed suspensions of water or easily volatile, organic solvents, such as e.g. of chloroform or chloroform-mixtures. Such a coating may be by dipping, spraying or dripping the suspension onto non-metallic or metallic surfaces of the implant, whereby they form coatings with a good adhesion.

Once applied to the implant, the coating may be dried in a gas stream or by the use of a vacuum and/or increased temperature. The coating may also be applied to a pre-warmed implant (e.g. where the implant is at a temperature of about 70° C. or more).

In one embodiment the coating is a coating which is present without an additional support or additional carrier. In other words, the coating essentially or even completely comprises only said composite salts. This significantly facilitates the production of such implants. Thus the suggested composite salts can be applied directly as a coating, without the need for an additional specific support or carrier.

In another embodiment, the coating may comprise a bisphosphonate and a water-soluble ionic polymeric component. The coating may further comprise an amphiphilic component.

The amphiphilic component, or the bisphosphonate and the water-soluble ionic polymeric component, respectively, are present as a mixture, preferably as a composite salt (i.e. the amphiphilic component is also ionic) with a low solubility in water. By using an amphiphilic or water-soluble ionic polymeric component, good adhesion of the bisphosphonate on implant materials is achievable.

In one embodiment the water-soluble ionic polymeric component, which in the composite salt with the bisphosphonate is the reason for a reduced solubility of the bisphosphonate, is a polymeric component with free anionic groups, preferably a polymeric component, which is derived from biologically compatible biopolymers. Thus, the water-soluble ionic polymeric component can be carboxylated, carboxymethylated, sulphated, or phosphorylated derivates of natural polysaccharides. In one embodiment, the water-soluble ionic polymeric component is a polysaccharide selected from dextran, pullulane, chitosan, starch, or cellulose, or mixtures thereof.

In one embodiment, the bisphosphonate (which may be an amino-bisphosphonate) and the amphiphilic component (which may be an alkyl-sulfate or alkyl-carboxylate), are present in the coating in a molar ratio of between about 10:1 and about 1:5. In one embodiment the molar ratio is about 2:1 to about 1:2. Accordingly, in a further embodiment, the bisphosphonate (such as an amino-bisphosphonate) and the water-soluble ionic polymeric component are present in the coating preferably in a molar ratio between about 10:1 and about 1:5, more preferably in a molar ratio from about 2:1 to about 1:2, each with respect to the amino groups of the amino group-containing bisphosphonate used and the anionic groups present in the polymeric component.

Such a coating can be applied to an even (smooth), porous and/or roughened surface. The surface structure can be produced by mechanical processes (e.g. sand blasting) and/or by chemical processes (e.g. acid treatment).

In one embodiment, the coating has a thickness in the range of about 0.1-about 10 μm, (i.e. about 0.2-about 8 μm, about 0.3-about 6 μm). In one embodiment, the coating has a thickness in the range of about 0.5-about 5 μm.

In one embodiment, the coating comprises a bisphosphonate at a concentration of about 0.1-about 100 μg/cm² (i.e. about 1-about 50 μg/cm², about 2-about 20 μg/cm² or about 5-about 10 μg/cm²). For example, in the experiments described in reference 5, alendronate was coated onto a dental implant at a concentration of 10 μg/cm².

In one embodiment, the implant is coated with about 0.1-about 50 μg bisphosphonate (i.e. about 1-about 25 μg bisphosphonate, about 2-about 10 μg bisphosphonate, about 4-about 6 μg bisphosphonate). For example, in the experiments described in reference 26, 2.1 μg zoledronate was calculated to be coated onto a 3×5 mm implant, while in reference 27, 3×5 mm titanium implants were coated with 0.2, 2.1, 8.5 or 16 μg zoledronate. In one embodiment, the implant is coated with 8.5 μg zoledronate. Such exemplary coating concentrations may be used in the methods and compositions of the instant disclosure.

Not all the bisphosphonate contained within a coating may be released into the surrounding tissues following implantation. Therefore, in one embodiment, the implant releases from its coating about 0.1 μg to about 50 μg bisphosphonate (i.e. about 1 μg to about 25 μg bisphosphonate, about 2 μg to about 10 μg bisphosphonate, about 4-about 6 μg bisphosphonate).

Two methods for determining the amount of bisphosphonate coated onto an implant are disclosed in reference 28, the contents of which are incorporated by reference. These methods calculated the amount of bisphosphonate coated onto an implant by subtraction, after measuring the residual concentration of bisphosphonate in the supernatant.

Depot formulations of zoledronic acid, as well as crystalline forms and salts of zoledronic acid useful in depot formulations, which may also be used in the instant disclosure, are provided in United States Published Patent Application Nos. 2010-0056481 and 2010-0047306, both of which are incorporated by reference herein in their entirety.

### Bone Anabolic Coating

As noted above, the bone anabolic agent may be formulated as a gel and then coated onto the implant prior to fixation.

If the bone anabolic agent is an antibody, such as an anti-sclerostin antibody, reconstitution to give an antibody concentration in a gel of at least about 50 mg/mL is typical e.g. >about 100 mg/mL, >about 150 mg/mL, >about 200 mg/mL, >about 250 mg/mL, etc.

Such gel formulations are typically turbid. For example, they may have a turbidity above about 500 NTU (Nephelometric Turbidity Units) e.g. ≧about 750 NTU, ≧about 1000 NTU, ≧about 1250 NTU, etc. when measured at 25° C. and atmospheric pressure. For example, a useful gel formulation of antibody Antibody 1 has a turbidity of about 1350 NTU.

Alternatively, the bone anabolic may be added to a coating on the implant during manufacture of the implant. For example, references 29 and 30 describe methods of coating implants, where a variety of actives may be included in the coating and are then released. These actives include antibodies. Furthermore, reference 31 discloses the use of a polyurethane hydrogel containing active antibodies for coating implants. Such a coating was able to release 14 μg/cm² IgG after 4 hours. Another hydrogel, this time made from hyaluronic acid, is disclosed in reference 32 which allows the release of bioactive IgG. Reference 33 discloses controlled antibody release from a matrix of poly(ethylene-co-vinyl acetate) (poly EVA), where the rate of release can be adapted depending on the molecular weight of the matrix used.

In one embodiment, the coating is a polymer coating comprising an anti-sclerostin antibody. In one embodiment, the coating comprises a hydrogel and an anti-sclerostin antibody. In another embodiment, the coating comprises poly EVA and an anti-sclerostin antibody.

In one embodiment, the implant is coated with lyophilised anti-sclerostin antibody.

In one embodiment, the implant is coated with about 0.01 mg to about 1000 mg (i.e. about 0.1-about 500 mg, about 1 mg to about 250 mg, about 2 mg to about 100 mg, about 5 mg to about 50 mg or about 10 mg to about 20 mg) anti-sclerostin antibody. The amount coated would depend on the size of the implant, the surface area of the implant and the thickness of the coating. The amount coated may also depend on the desired application of the implant as well as the health of the patient (e.g., do they suffer from low bone mineral density).

PTH may be used in an implant coating [34]. If PTH is used, it may be applied as part of a polyethylene glycol matrix (e.g., as a gel). In one embodiment, the implant coating comprises PTH at a concentration of about 1 μg/ml to about 50 μg/ml (e.g., about 5 μg/ml to about 40 μg/ml PTH, about 10 μg/ml to about 30 μg/ml PTH). In one embodiment, the implant coating comprises PTH at a concentration of about 20 μg/ml.

### Patient Groups

In one embodiment, the patient being treated has a fracture to a limb (i.e., leg or arm) or joint (e.g., knee or hip). Thus, in one embodiment, the patient being treated has a fracture to one or more of the humerus, skull, pelvis, radius, ulnar, a carpal, a metacarpal, the clavical, scapular, femur, os coxae, patella, tibia, fibula, talus, calcaneus, a tarsal, a metatarsal, the ischium or the ileum. In another embodiment, the patient being treated has undergone, or will undergo surgery on one or more of the following joints: knee, hip, ankle, shoulder, elbow. Such surgery includes hip replacement and knee replacement. In one embodiment, the patient has a spinal injury or deformation due to illness or genetic disease. In one embodiment, the patient is one who requires spinal fusion surgery.

In another embodiment, the patient being treated has received or will receive a dental implant.

In one embodiment, the patient being treated is one who has been identified as being at risk of suffering from osteoporosis. In one embodiment, the patient being treated has osteoporosis (including steroid-induced osteoporosis and male osteoporosis). In one embodiment, the patient has a bone metabolic disease leading to low bone mass (BM) development and/or fractures. In one embodiment, the patient being treated is one who has osteogenesis imperfecta or hypophosphatasia. These embodiments include both (i) patients at risk of fractures, and (ii) patients not at risk of fractures. Such a patient may be identified by looking at, for example, nutritional intake, family history, genetic markers, medical examination, serological bone biomarkers, and bone mineral density by DEXA, and overall fracture assessment by FRAX™.

In one embodiment, the patient is a less than 5 years old, 5-10 years old, 10-20 years old, 20-30 years old, or 30-40 years old. In one embodiment, the patient is 40 years of age or older, 50 years of age or older, 60 years of age or older, or 70 years of age or older.

In one embodiment, the patient is a post-menopausal woman.

### Kits

In one embodiment, the disclosure provides kits comprising a bone implant, a bone anabolic agent, a bone resorption inhibitor and instructions for use.

One or both of the bone anabolic agent and the bone resorption inhibitor may be provided in lyophilised form and the kit may further comprise a diluent and instructions for use.

Such kits may optionally further comprise infusion bags or syringes in order to administer the bone anabolic agent and bone resorption inhibitor.

In a further embodiment, the disclosure provides a kit comprising: (i) a bone implant coated with a bone anabolic agent, (ii) a bone resorption inhibitor for systemic administration, and (iii) instructions for use.

In a further embodiment, the disclosure provides a kit comprising: (i) a bone implant coated with a bone resorption inhibitor, (ii) a bone anabolic agent for systemic administration, and (iii) instructions for use.

In a further embodiment, the disclosure provides a kit comprising: (i) a bone implant coated with a bone anabolic agent, (ii) a bone resorption inhibitor for local administration, and (iii) instructions for use.

In a further embodiment, the disclosure provides a kit comprising: (i) a bone implant coated with a bone resorption inhibitor, (ii) a bone anabolic agent for local administration, and (iii) instructions for use.

In a further embodiment, the disclosure provides a kit comprising: (i) a bone implant coated with a bone resorption inhibitor and a bone anabolic agent, and (ii) instructions for use.

### Combination Packages

Combination packages are those where the implant and active ingredients are provided in a single sterile package which allows coating of the implant with the active ingredients prior to delivery. Examples of such combination packages are described in reference 35.

In one embodiment, the disclosure provides a combination package comprising a bone anabolic agent, a bone resorption inhibitor and an implant. The implant may be a dental implant. In one embodiment, the disclosure provides a combination package comprising a bone anabolic agent, a bone resorption inhibitor and an implant, wherein the bone resorption inhibitor is pre-coated on the implant and the bone anabolic agent is provided as a solution ready for coating onto the implant. In one embodiment, the disclosure provides a combination package comprising a bone anabolic agent, a bone resorption inhibitor and an implant, wherein the bone anabolic agent is pre-coated on the implant and the bone resorption inhibitor is provided as a solution ready for coating onto the implant.

In one embodiment, the disclosure provides a combination package comprising a bone anabolic agent, a bone resorption inhibitor and an implant, wherein the bone anabolic agent is pre-coated on the implant in lyophilised form and the bone resorption inhibitor is provided as a solution ready for coating onto the implant. In such an embodiment, the bone resorption inhibitor solution also reconstitutes the lyophilised bone anabolic agent. In such an embodiment, the bone anabolic agent may be an anti-sclerostin antibody such as Antibody 1.

Such a combination package will typically further comprise instructions for use.

### General

The term “comprising” means “including” as well as “consisting” e.g. a composition “comprising” X may consist exclusively of X or may include something additional e.g. X+Y.

The term “about” in relation to a numerical value x means, for example, x±10%.

### BRIEF DESCRIPTION OF DRAWINGS

FIG. 1 discloses removal torque values (in N-mm) 2 weeks post-implantation (n=8/group). Group 1=ovariectomy (OVX) group receiving control implant, 2=OVX group receiving zoledronic acid coated implant; 3=OVX group receiving control implant and weekly intravenous anti-sclerostin antibody treatment; 4=OVX group receiving zoledronic acid coated implant and weekly intravenous anti-sclerostin antibody treatment; 5=Intact group receiving control implant; Mean±SEM, ANOVA, Dunnett, ** p<0.01 versus OVX control (group 1).

FIG. 2 discloses removal torque values (in N-mm) 4 weeks post-implantation (n=8/group). Group 1=OVX group receiving control implant, 2=OVX group receiving zoledronic acid coated implant; 3=OVX group receiving control implant and weekly intravenous anti-sclerostin antibody treatment; 4=OVX group receiving zoledronic acid coated implant and weekly intravenous anti-sclerostin antibody treatment; 5=Intact group receiving control implant; ANOVA, Dunnett, * p<0.05, ** p<0.01 versus OVX control; x p<0.05 single treatment versus combination treatment.

### MODES FOR CARRYING OUT THE DISCLOSED METHODS AND IMPLANTS

### Example 1

Titanium screw type implants (3 mm length, 1-1.5 mm diameter, self-cutting) were prepared by either (1) sand blasting and acid etching with no further coating, or (2) sand blasted and acid etched, then coated with 8.5 μg zoledronate.

The coating was carried out by warming the implants and then dip coating with a zolendronate stearate salt and then allowing to dry at 80° C. as described for alendronic acid coating in reference 36. The spraying and drying cycle was carried out 3 times.

Skeletally mature virgin Wistar rats (6.5-month-old, Harlan laboratories, Switzerland) were estrogen-deprived by ovariectomy (OVX) under narcosis. Bone mineral density loss was confirmed in the proximal tibia metaphysis (4.5 mm distal from proximal end) three months post-ovariectomy (compared to intact controls) by peripheral quantitative computed tomography as described previously [37]. The titanium screws were implanted approximately 3 mm distal to the proximal end of the left tibia under narcosis. Animals received a sand-blasted acid edged titanium implant with or without zoledronic acid coating which were prepared as above. Animals were distributed into the following groups [n=16/group and time point]:

1. OVX group receiving control implant
2. OVX group receiving zoledronic acid coated implant
3. OVX group receiving control implant and weekly intravenous (iv.) anti-sclerostin antibody Antibody 1 (100 mg/kg)
4. OVX group receiving zoledronic acid coated implant and weekly iv. anti-sclerostin antibody Antibody 1 (100 mg/kg)
5. Intact group receiving control implant

Animals were sacrificed 2 and 4 weeks post-implantation. The left tibiae was excised for histomorphometric and micro computed tomography based evaluations of osseointegration (n=8) and biomechanical removal torque testing (n=8) as described previously [38, 39].

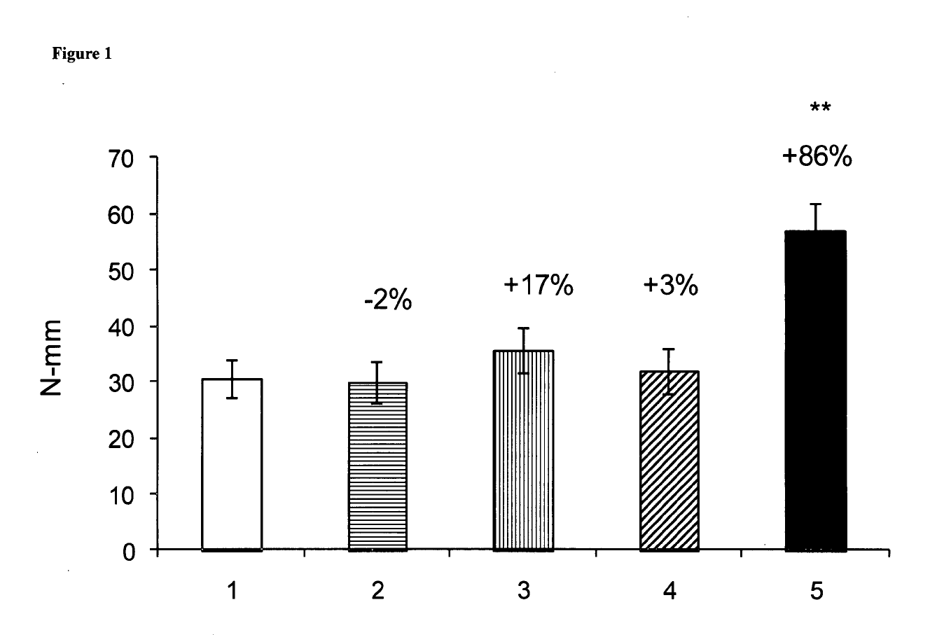

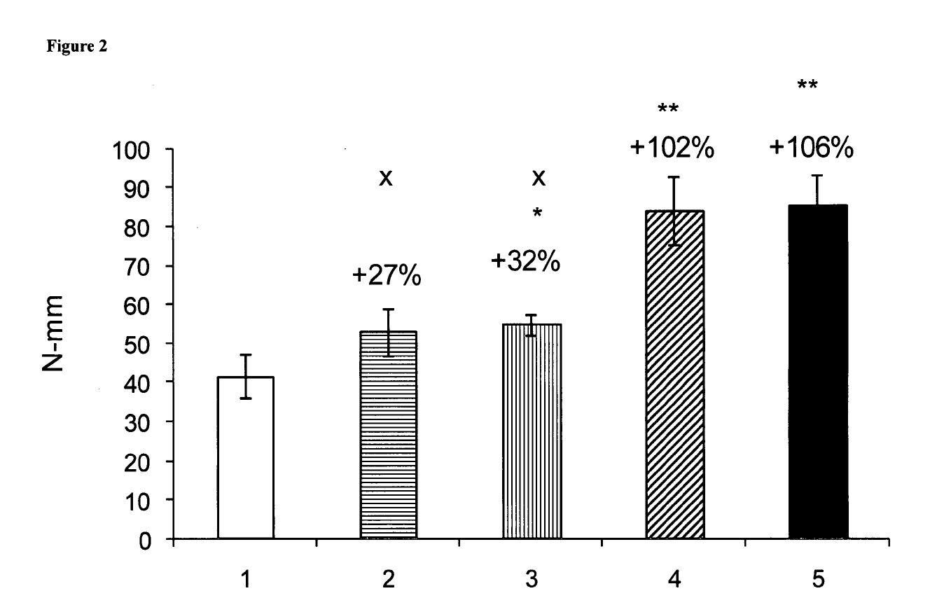

Removal torque was comparable between OVX groups two weeks post-implantation (group 1-4, FIG. 1). As expected removal torque was substantially higher (+86%) in intact animals, which had not experienced OVX induced bone loss (group 5, FIG. 1). Four weeks post-implantation removal torque was non-significantly increased by 27% in the animals having received a zoledronic acid coated implant (group 2, FIG. 2). Animals having been exposed to weekly iv. anti-sclerostin antibody treatment displayed a significant increase of 32% (group 3, FIG. 2). The combination of zoledronic acid coated implant with anti-sclerostin antibody treatment resulted in an increase in removal torque up to the level of the intact control (group 4+102% and group 5 106% respectively, FIG. 2). Removal torque was significantly higher in the group receiving the combination (group 4) compared to single treatment (groups 2 and 3).

It will be understood that the disclosed methods and implants has been described by way of example only and modifications may be made whilst remaining within the scope and spirit of the disclosed methods and implants.

### REFERENCES

### The Contents of which are Hereby Incorporated in Full

[1] US20100094426
[2] B. Sanden 2001, Fixation of Spinal Implants: Clinical and Experimental Studies on the Effects of Hydroxyapatite Coating, Dissertation for the Degree of Doctor of Philosophy (Faculty of Medicine) in Orthopaedics presented at Uppsala University, Sweden, 2001.
[3] Branemark et al. 2001, J. Rehab. Res. and Dev. 38(2):175-181.
[4] Procter. 2009, European Cells and Materials. 17, Suppl. 1:4.
[5] Langhoff et al. 2008, Int. J. Oral Maxillofac. Surg., 37:1125-1132.
[6] WO00/32773
[7] WO2005/014650
[8] WO2005/003158
[9] WO2006/119107
[10] WO2008/061013
[11] WO2008/133722
[12] WO2008/115732
[13] U.S. Pat. No. 7,592,429 B2
[14] WO2009/047356
[15] WO2009/131553
[16] Titanium in Medicine, Material Science, Surface Science, Engineering, Biological Responses and Medical Applications Series: Engineering Materials. Brunette, D. M. et al. Eds.
[17] Buser et al. 2004 J. Den. Res. 83(7):529-533.
[18] WO2007/040010
[19] WO2008/081023
[20] EP0875231
[21] WO97/37628
[22] Shaw & Bishop. 2005, Arch. Dis. Child, 90:494-499
[23] PCT/EP2010/052665
[24] US2009/0130177
[25] Greiner et al. 2008, Acta Orthopaedica, 79(5):717-725.
[26] Stadelmann et al. 2008, European Cells and Materials, 16:10-16.
[27] Peter et al. 2005, JBMR online. DOI: 10.1002/jbm.a.30456.
[28] Josse et al. 2004, Advanced Materials, 16(16):1423-1427.
[29] U.S. Pat. No. 6,844,024
[30] WO00/15273
[31] Rojas et al. 2000, J. Controlled Release 63:175-189.
[32] Tian et al. 2005, J. Controlled Release, 102:13-22.
[33] Saltzman et al. 1993, J. Applied Polymer Science, 48:1493-1500.
[34] Jung et al. 2007, Clin. Oral Impl. Res. 18:319-325.
[35] WO2009/147166
[36] US2008/0286328
[37] Keller & Kneissel, 2005, Bone, 37:148-58.
[38] Ferguson et al. 2008, Int J Oral Maxillofac Implants, 23:1037-46.
[39] Schliephake et al. 2010, J Clin Peridontol doi: 10.1111/j.1600-051X.2010.01549

1-20. (canceled)

21. A method for improving the osseointegration of a bone implant, comprising, implanting zoledronic acid-coated implant in a patient in need thereof and administering an anti-sclerostin antibody to the patient, said antibody comprising a variable heavy (V_{H}) domain comprising the three complementarity determining regions (CDRs) set forth as SEQ ID NOs: 3, 4 and 5 and a variable light (V_{L}) domain comprising the three CDRs set for as SEQ ID NOs: 6, 7 and 8.

22. The method of claim 21, wherein the anti-sclerostin antibody is administered to the patient before or after the implant is implanted in the patient.

23. The method of claim 21, wherein the implant is a dental implant, a bone plate, bone screw, spinal implant or replacement joint, including, but not limited to knee, hip, ankle, shoulder, elbow, wrist and knuckle joints.

24. The method of claim 21, wherein the anti-sclerostin antibody is administered to the patient at a dose of between about 20 mg/kg to about 100 mg/kg.

25. The method of claim 21, wherein the antibody comprises as V_{H} domain comprising the amino acid sequence set forth as SEQ ID NO: 1 and a V_{L} domain comprising the amino acid sequence set forth as SEQ ID NO: 2.

26. A method for improving the osseointegration of a bone implant in a patient, comprising, systemically administering an anti-sclerostin antibody to a patient having a zoledronic acid-coated bone implant, said antibody comprising a variable heavy (V_{H}) domain comprising the three complementarity determining regions (CDRs) set forth as SEQ ID NOs: 3, 4 and 5 and a variable light (V_{L}) domain comprising the three CDRs set for as SEQ ID NOs: 6, 7 and 8.

27. The method of claim 26, wherein the implant is a dental implant, a bone plate, bone screw, spinal implant or replacement joint, including, but not limited to knee, hip, ankle, shoulder, elbow, wrist and knuckle joints.

28. The method of claim 26, wherein the anti-sclerostin antibody is administered to the patient at a dose of between about 20 mg/kg to about 100 mg/kg.

29. The method of claim 26, wherein the antibody comprises as V_{H} domain comprising the amino acid sequence set forth as SEQ ID NO: 1 and a V_{L} domain comprising the amino acid sequence set forth as SEQ ID NO: 2


SEQUENCE LISTING (10 sequences)

SEQ ID NO: 1 - protein, 117 aa, artificial
  Antibody Vh
QVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSS

SEQ ID NO: 2 - protein, 113 aa, artificial
  Antibody Vl
DIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQ

SEQ ID NO: 3 - protein, 10 aa, Artificial
  CDR
GFTFRSHWLS

SEQ ID NO: 4 - protein, 20 aa, Artificial
  CDR
WVSNINYDGSSTYYADSVKG

SEQ ID NO: 5 - protein, 8 aa, artificial
  CDR
DTYLHFDY

SEQ ID NO: 6 - protein, 14 aa, Artificial
  CDR
TGTSSDVGDINDVS

SEQ ID NO: 7 - protein, 11 aa, artificial
  CDR
LMIYDVNNRPS

SEQ ID NO: 8 - protein, 10 aa, artificial
  CDR
QSYAGSYLSE

SEQ ID NO: 9 - protein, 462 aa, artificial
  Antibody heavy chain
MAWVWTLPFLMAAAQSVQAQVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 237 aa, artificial
  Antibody light chain
MSVLTQVLALLLLWLTGTRCDIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS